(19) United States
(12) Patent Application Publication
(10) Pub. No.: US 2014/0277388 A1
(43) Pub. Date: Sep. 18, 2014

(54) BIOCOMPATIBLE FOAM OCCLUSION DEVICE FOR SELF-EXPANDING HEART VALVES

(71) Applicant: AGA MEDICAL CORPORATION, Plymouth, MN (US)

(72) Inventor: Benedict Skemp, Golden Valley, MN (US)

(73) Assignee: AGA MEDICAL CORPORATION, Plymouth, MN (US)

(21) Appl. No.: 13/797,466

(22) Filed: Mar. 12, 2013

(51) Int. Cl. A61F 2/24

(52) U.S. Cl. CPC A61F 2/2442
USPC 623/1.26; 623/2.37; 623/2.11

(57) ABSTRACT

A self-expandable ring seals a gap between a medical device and adjacent body tissue. The ring is disposed about the medical device and configured to self-expand from a first radius in a compressed condition to a second radius in an expanded condition, the second radius being greater than the first radius. The ring may be formed from a compliant biocompatible foam configured and arranged to conform to the body tissue.

### BACKGROUND OF THE INVENTION

The present disclosure relates in general to heart valve replacement and, in particular, to collapsible prosthetic heart valves. More particularly, the present disclosure relates to devices and methods for positioning collapsible prosthetic heart valves and sealing same in the patient's anatomy to minimize or prevent perivalvular leakage.

Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible prosthetic heart valves typically take the form of a valve structure mounted on a stent. There are two types of stents on which the valve structures are ordinarily mounted: a self-expanding stent or a balloon-expandable stent. To place such valves into a delivery apparatus and ultimately into a patient, the valve must first be collapsed or crimped to reduce its circumferential size.

When a collapsed prosthetic valve has reached the desired implant site in the patient (e.g., at or near the annulus of the patient's native heart valve that is to be replaced by the prosthetic valve), the prosthetic valve can be deployed or released from the delivery apparatus and re-expanded to full operating size. For balloon-expandable valves, this generally involves releasing the entire valve, and then expanding a balloon positioned within the valve stent. For self-expanding valves, on the other hand, the stent automatically expands as the sheath covering the valve is withdrawn.

### SUMMARY OF THE INVENTION

In one example, a biocompatible foam structure for sealing a gap between a medical device and adjacent body tissue includes an expandable ring-shaped body. The expandable ring-shaped body may be configured to be disposed about the medical device and to expand from a first radius in a compressed condition to a second radius in an expanded condition, the second radius being greater than the first radius. The body may be formed from a compliant biocompatible configured and arranged to conform to the body tissue.

A prosthetic heart valve includes a collapsible and expandable stent having a proximal end, a distal end, an annulus section adjacent the proximal end and an aortic section adjacent the distal end, a collapsible and expandable valve assembly disposed within the stent and including a plurality of leaflets, and a cuff annularly disposed about the valve assembly in the annulus section. The heart valve may further include a ring-shaped body disposed about the stent and configured to expand from a first radius in a compressed condition to a second radius in an expanded condition, the second radius being greater than the first radius, the body being formed from a compliant biocompatible foam configured to conform to body tissue.

In some examples, a method of sealing a space between a medical device and adjacent tissue includes delivering the medical device and a ring-shaped body to a target site using a delivery system having a sheath disposed over the medical device and the body. The sheath may be removed to expose the medical device and the body such that the medical device and the body expand. The space between the expanded medical device and the adjacent tissue may be sealed with the expanded body disposed therebetween.

### BRIEF DESCRIPTION OF THE DRAWINGS

Various embodiments of the present disclosure are described herein with reference to the drawings, wherein:

FIG. 1 is a side elevational view of a collapsible prosthetic heart valve;

FIG. 2 is a highly schematic cross-sectional view taken along line A-A of FIG. 1 and showing the prosthetic heart valve disposed within a native valve annulus;

FIG. 3A is a perspective view of an exemplary foam;

FIG. 3B is a diagrammatic view of the foam of FIG. 3A after it has been die cut into a cylindrical shape;

FIG. 3C is a diagrammatic view of the cylindrical foam of FIG. 3B after it has been cut into a ring configuration, the foam ring being in a relaxed configuration;

FIG. 3D is a perspective view of the foam ring of FIG. 3C in a deformed condition;

FIG. 3E is a side elevational view of the prosthetic heart valve of FIG. 1 with the foam ring of FIGS. 3C and 3D disposed about the annulus section thereof;

FIGS. 4A-D illustrate the steps used to deploy a prosthetic heart valve having a foam ring within a native valve annulus; and

FIG. 5 is a highly schematic cross-sectional view showing a prosthetic heart valve disposed with a foam ring in an expanded state within a native valve annulus.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to the collapsible prosthetic heart valve delivery process, conventional devices suffer from some shortcomings. For example, with conventional self-expanding valves, clinical success of the valve is dependent on accurate deployment and effective sealing within the patient's anatomy. Inaccurate deployment and anchoring may result in the leakage of blood between the implanted heart valve and the native valve annulus, commonly referred to as paravalvular (also sometimes referred to as perivalvular) leakage. In aortic valves, this leakage enables blood flow from the aorta back into the left ventricle, reducing cardiac efficiency and putting a greater strain on the heart muscle. Additionally, calcification of the aortic valve may affect performance and the interaction between the implanted valve and the calcified tissue is believed to be relevant to leakage.

Moreover, anatomical variations between patients may require removal of a fully deployed heart valve from the patient if it appears that the valve is not functioning properly. Removing a fully deployed heart valve increases the length of the procedure and increases the risk of infection and/or damage to heart tissue. Thus, methods and devices are desirable that would reduce the need to remove a deployed valve. Methods and devices are also desirable that would reduce the likelihood of paravalvular leakage around the implanted heart valve.

There therefore is a need for further improvements to the devices, systems, and methods for transcatheter positioning of collapsible prosthetic heart valves and the sealing of the implanted valves within the patient's anatomy. Specifically, there is a need for further improvements to the devices, systems, and methods for sealing a prosthetic heart valve within a native valve annulus. Among other advantages, the present disclosure may address one or more of these needs.

As used herein, the term “proximal,” when used in connection with a prosthetic heart valve, refers to the end of the heart valve closest to the heart when the heart valve is implanted in a patient, whereas the term “distal,” when used in connection with a prosthetic heart valve, refers to the end of the heart valve farthest from the heart when the heart valve is implanted in a patient. When used in connection with devices for delivering a prosthetic heart valve or other medical device into a patient, the terms “trailing” and “leading” are to be taken as relative to the user of the delivery devices. “Trailing” is to be understood as relatively close to the user, and “leading” is to be understood as relatively farther away from the user.

The biocompatible foams of the present invention may be used in connection with collapsible prosthetic heart valves. FIG. 1 shows one such collapsible stent-supported prosthetic heart valve 100 including a stent 102 and a valve assembly 104 as known in the art. Prosthetic heart valve 100 is designed to replace a native tricuspid valve of a patient, such as a native aortic valve. It should be noted that while the inventions herein are described predominately in connection with their use with a prosthetic aortic valve and a stent having a shape as illustrated in FIG. 1, the valve could be a bicuspid valve, such as the mitral valve, and the stent could have different shapes, such as a flared or conical annulus section, a less-bulbous aortic section, and the like, and a differently shaped transition section.

Prosthetic heart valve 100 will be described in more detail with reference to FIG. 1. Prosthetic heart valve 100 includes an expandable stent 102 which may be formed from, for example, a shape memory material, such as the nickel-titanium alloy known as “Nitinol” or other suitable metals. and in particular, from those materials that are capable of self-expansion. Stent 102 extends from a proximal or annulus end 130 to a distal or aortic end 132, and includes an annulus section 140 adjacent proximal end 130, as well as transition section 141 and aortic section 142 adjacent distal end 132. Annulus section 140 has a relatively small cross-section in the expanded condition, while aortic section 142 has a relatively large cross-section in the expanded condition. Preferably, annulus section 140 is in the form of a cylinder having a substantially constant diameter along its length. Transition section 141 may taper outwardly from annulus section 140 to aortic section 142. Each of the sections of stent 102 includes a plurality of struts 160 forming cells 162 connected to one another in one or more annular rows around stent 102. For example, as shown in FIG. 1, annulus section 140 may have two annular rows of complete cells 162 and aortic section 142 and transition section 141 may each have one or more annular rows of partial cells 162. Cells 162 in aortic section 142 may be larger than the cells in annulus section 140. The larger cells in aortic section 142 better enable prosthetic valve 100 to be positioned in the native valve architecture without the stent structure interfering with blood flow to the coronary arteries.

Stent 102 may also include a plurality of commissure features 166 for attaching the commissure between two adjacent leaflets to stent 102. As can be seen in FIG. 1, commissure features 166 may lie at the intersection of four cells 162, two of the cells being adjacent one another in the same annular row, and the other two cells being in different annular rows and lying in end-to-end relationship. Preferably, commissure features 166 are positioned entirely within annulus section 140 or at the juncture of annulus section 140 and transition section 141. Commissure features 166 may include one or more eyelets which facilitate the suturing of the leaflet commissure to stent 102.

Stent 102 may include one or more retaining elements 168 at distal end 132 thereof, retaining elements 168 being sized and shaped to cooperate with female retaining structures (not shown) provided on the deployment device. The engagement of retaining elements 168 with the female retaining structures on the deployment device helps maintain prosthetic heart valve 100 in assembled relationship with the deployment device, minimizes longitudinal movement of the prosthetic heart valve relative to the deployment device during unsheathing or resheathing procedures, and helps prevent rotation of the prosthetic heart valve relative to the deployment device as the deployment device is advanced to the target location and the heart valve deployed.

Valve assembly 104 is secured to stent 102, preferably within annulus section 140 of stent 102. Valve assembly 104 includes cuff 176 and a plurality of leaflets 178 which collectively function as a one-way valve by coapting with one another. As a prosthetic aortic valve, valve 100 has three leaflets 178, as well as three commissure features 166. However, it will be appreciated that other prosthetic heart valves with which the leak occluders of the present invention may be used may have a greater or lesser number of leaflets 178 and commissure features 166.

Although cuff 176 is shown in FIG. 1 as being disposed on the lumenal or inner surface of annulus section 140, it is contemplated that cuff 176 may be disposed on the ablumenal or outer surface of annulus section 140 or may cover all or part of either or both of the lumenal and ablumenal surfaces. Both cuff 176 and leaflets 178 may be wholly or partly formed from any suitable biological material, such as porcine or bovine pericardial tissue, or from a polymer such as, for example, polytetrafluoroethylene.

Prosthetic heart valve 100 may be used to replace a native aortic valve, a surgical heart valve or a heart valve that has undergone a surgical procedure. Prosthetic heart valve 100 may be delivered to the desired site (e.g., near the native aortic annulus) using any suitable delivery device. During delivery, prosthetic heart valve 100 is disposed inside the delivery device in the collapsed condition. The delivery device may be introduced into a patient using a transfemoral, transapical, transseptal or any other percutaneous approach. Once the delivery device has reached the target site, the user may deploy prosthetic heart valve 100. Upon deployment, prosthetic heart valve 100 expands so that annulus section 140 is in secure engagement within the native aortic annulus. When prosthetic heart valve 100 is properly positioned inside the heart, it works as a one-way valve, allowing blood to flow from the left ventricle of the heart to the aorta, and preventing blood from flowing in the opposite direction.

Problems may be encountered when implanting prosthetic heart valve 100. For example, in certain procedures, collapsible valves may be implanted in a native valve annulus without first resecting the native valve leaflets. The collapsible valves may have clinical issues because of the nature of the stenotic leaflets that are left in place. Additionally, patients with uneven calcification, bi-cuspid aortic valve disease, and/or valve insufficiency cannot be treated well, if at all, with the current collapsible valve designs.

The reliance on unevenly calcified leaflets for proper valve placement and seating could lead to several problems, such as paravalvular leakage (PV leak), which can have adverse clinical outcomes. To reduce these adverse events, the optimal valve would anchor adequately and seal within the native valve annulus without the need for excessive radial force that could harm nearby anatomy and physiology.

FIG. 2 is a highly schematic cross-sectional illustration of prosthetic heart valve 100 disposed within native valve annulus 250. As seen in the figure, annulus section 140 of stent 102 has a substantially circular cross-section which is disposed within non-circular native valve annulus 250. At certain locations around the perimeter of heart valve 100, crescent-shaped gaps 200 form between the heart valve and native valve annulus 250. Blood flowing through these gaps and around leaflets 178 of valve assembly 104 can cause regurgitation and other inefficiencies which reduce cardiac performance. Such improper fitment may result from suboptimal native valve annulus geometry due, for example, to calcification of native valve annulus 250 or to unresected native leaflets.

FIG. 3A illustrates a biocompatible foam 300 in the shape of body 310. The term “foam” is used herein in an inclusive sense to refer to foamed plastic materials (also sometimes called “cellular plastics”, “cellular polymers”, “plastic foams” or “expanded plastics”) and more specifically refers to plastic materials in which the apparent density is decreased by the presence of numerous voids disposed throughout its mass. Additionally, body 310 of biocompatible foam 300 may be formed of a polymeric shape-memory material or other suitable material that has the ability to return from a deformed or compressed state to its original or expanded shape. Alternatively, body 310 may be formed of any other compressible foam or material.

Biocompatible foam 300 may be activated using a stimulus, such as a chemical stimulus, light or heat. For example, biocompatible foam 300 may be a heat-activated shape-memory foam capable of changing shape due to a change in temperature such as, for example, by being brought in contact with a warm saline injection or natural body heat. The temperature at which foam 300 changes from a deformed shape to its original shape or vice versa is referred to as the transition temperature. In examples in which a heat-activated shape-memory foam is used, biocompatible foam 300 may have a transition temperature ranging from about 30 degrees Celsius to about 50 degrees Celsius. In some examples, biocompatible foam 300 may have a transition temperature from about 24 degrees Celsius to about 45 degrees Celsius. In some other examples, biocompatible foam 300 may have a transition temperature ranging from about 35 degrees Celsius to about 39 degrees Celsius. In some examples, biocompatible foam 300 may have a transition temperature that is within one degree Celsius from normal core body temperature (i.e., about 98.6° F.). An example of a suitable heat-activated shape-memory foam is SMP Foam, available from SMP Technologies Inc. (Tokyo, Japan).

Another parameter that may be useful in choosing the proper biocompatible foam 300 is the density of the foam. As previously noted, a foam typically includes voids dispersed throughout its mass, which will decrease the density of the foam. The density of biocompatible foam 300 may be high enough to impede blood flow, but low enough to permit adequate compression such that the foam may be delivered to the target site via a low profile delivery system (e.g., 18 Fr delivery system) and to allow the heart valve to fully expand therein. In one example, biocompatible foam 300 may have a density between about 10 kg/m³ and about 60 kg/m³. In other examples, biocompatible foam 300 may have a density between about 45 kg/m³ and about 55 kg/m³.

Biocompatible foam 300 may also be chosen based on the volume change between the compressed state and the expanded state. For example, biocompatible foam 300 may experience a volume change of between about 500% and about % 1000 when subjected to its transition temperature or other means of transition. For example, foam 300 may experience a volume change of about 700% when its temperature is raised from 35 degrees Celsius to 37 degrees Celsius.

As seen in FIG. 3B, body 310 of biocompatible foam 300 may be cut into a disk having a first radius r1 using a first punch die P1 or any suitable method. Second punch die P2 having a smaller diameter than punch die P1 may be used to remove inner material to form lumen 320 in the disk and thereby create foam ring 330 as shown in FIG. 3C. Ring 330 may have a first radius r1 in its fully-expanded state (FIG. 3C) and a second radius r2 in a deformed or contracted state (FIG. 3D). For example, ring 330 may have a first radius of about 17 mm in its fully-expanded state and may be deformed to a second radius of about 10 mm, which may be further compressed to a radius of about 1.75 mm for delivery and implantation. Moreover, as will be appreciated from FIGS. 3C-3D, lumen 320 may also expand and contract, allowing ring 330 to accommodate an expanding heart valve, as will be shown with reference to FIG. 3E.

The size, shape and density of ring 330 may be adjusted to achieve a desirable profile of radial forces. For example, ring 330 may be constructed such that when fully expanded it has a circumferential stress of 350 kPa. Additionally, as will be appreciated from FIG. 3E, ring 330 will exert a radial force against the supporting stent structure 102 disposed within its lumen 320. In this regard, excess radial force may cause the stent structure to collapse or prevent the stent from fully expanding within the native valve annulus, impeding or reducing the efficiency of the valve assembly. Inadequate radial forces on the other hand may prevent biocompatible foam from properly sealing the gaps between the walls of the native valve annulus and the heart valve such that paravalvular leakage is not reduced or prevented (i.e., blood will continue to flow around the heart valve).

One example of initializing shape-memory foam ring 330 for usage includes compressing the size of the ring after cutting it into the proper shape. Specifically, after heating ring 330 above the transition temperature of foam 300 or otherwise coaxing foam 300 to its expanded state (see r1 in FIG. 3C), the ring may be slowly compressed around a mandrel. Once ring 330 has been compressed as desired, it may be removed from the mandrel and quickly blown with compressed air, rapidly dropping the temperature of the ring and locking it into the compressed state. It is to be appreciated that though this technique has been described with reference to heat-activation, other methods of compressing foam 300 may also be used, such as light (e.g., UV light activation) or chemical activation.

The compressed ring 330 may be placed around the outer circumference of a prosthetic heart valve 100 and attached to heart valve 100 as shown in FIG. 3E. Lumen 320 of ring 330 may be sized so that the ring will be disposed around heart valve 100 near the transition section 141 with a friction fit and no additional attachments. Alternatively, ring 330 may be attached to selected struts 160, cuff 176, commissure features 166 or any other feature or combination of features of prosthetic heart valve 100. Ring 330 may be attached to features of prosthetic heart valve 100 via sutures, a biocompatible glue or adhesive, staples, clips or other suitable methods. Moreover, while FIG. 3E illustrates ring 330 of biocompatible foam 300 disposed about prosthetic heart valve 100 at the position of leaflets 178 in the axial direction, it will be understood that the ring may be arranged at any axial position along stent 102 and that multiple biocompatible foam rings may be utilized. In at least some examples, at least one of rings 330 overlies a portion of cuff 176. Moreover, as seen in FIG. 3E, ring 330 forms a foam band about prosthetic heart valve 100 for producing a seal with the native heart tissue. For example, ring 330 may extend about 3 mm to about 5 mm radially outward from the outer circumference of stent 102 when no external force is applied thereupon. As will be described in more detail below, ring 330 allows for superior sealing between the perimeter of heart valve 100 and native valve annulus 250, filling irregularities or gaps 200, while affording a low radial outward force.

FIGS. 4A-D illustrate the steps used to deploy ring 330 to seal prosthetic heart valve 100 within native valve annulus 250. While these figures illustrate transfemoral delivery of a prosthetic valve, it will be understood that ring 330 may be delivered via a transapical, transseptal or any other percutaneous approach. Additionally, ring 330 may be deployed to seal gaps in other locations as well, such as, for example, between the mitral valve and its surrounding tissue.

In a first step, heart valve 100, with ring 330 disposed thereabout, may be loaded into a delivery system 400 having an outer sheath 410, a distal cap 420 and an inner core 430. As shown in FIG. 4A, heart valve 100 may be disposed about core 430 and housed within outer sheath 410. Delivery system 400 may be advanced from the femoral artery, through the common iliac artery to the aorta until reaching native valve annulus 250, as shown by arrow S1 (FIG. 4A). If either heart valve 100 or delivery system 400 includes echogenic materials, such materials may be used to guide the delivery system to the appropriate position using the assistance of three-dimensional echocardiography to visualize the heart valve within the patient.

Upon reaching native valve annulus 250, delivery systems 400 may be distally advanced until distal cap 400 is positioned at a point past the native valve annulus, and outer sheath 410 may be proximally pulled back in the direction of arrow S2 to expose heart valve 100 (FIG. 4B). Self-expanding heart valve 100 may then begin to expand within native valve annulus 250. Foam ring 330 may also begin to expand upon deployment of heart valve 100 (e.g. due to the higher temperature within the patient's body)(FIG. 4C). Alternatively, ring 330 may be flushed with a warm saline solution, other reagents, or otherwise coaxed to promote radial expansion of the foam. In examples in which foam ring 330 is not made of a shape-memory material, it may be compressed within sheath 410 and the removal of the sheath may allow the compressed ring to expand to its natural state. Expansion of ring 330 will cause it to fill gaps 200 disposed between heart valve 100 and the walls of native valve annulus 250 and seal the heart valve within native valve annulus 250.

If heart valve 100 fails to perform adequately, for example, due to inadequate coaptation of the leaflets or improper placement, heart valve 100 may be retrieved within sheath 410 and repositioned and/or removed. To this end, various methods may be used to recapture a partially-deployed valve, such as, for example, through the use of tethers, clips or the like. Once heart valve 100 and foam ring 330 have been properly positioned and fully deployed and expanded, sheath 410 and distal cap 420 may be brought together and delivery system 400 may be proximally pulled through the center of the heart valve in the direction of arrow S2 and removed from the patient's body (FIG. 4C).

As seen in FIG. 4D, the expanded foam of ring 330 may be compliant and may conform to the surrounding walls of native valve annulus 250. In this figure, heart valve 100 has been implanted in a patient with the annulus portion 140 thereof positioned in native valve annulus 250 and gaps 200 between the heart valve and the native valve annulus have been substantially sealed by foam ring 330.

FIG. 5 is a highly schematic cross-sectional view showing the biocompatible foam of ring 330 in its relaxed state and fully radially expanded to fill crescent-shaped gaps 200 between heart valve 100 and native valve annulus 250. The foam 300 of ring 330 may also be capable of promoting tissue growth between heart valve 100 and native valve annulus 250. For example, foam 300 may be treated with a biological or chemical agent to promote tissue growth on ring 330, further sealing heart valve 100 within native valve annulus 250. Alternatively, foam 300 may be sufficiently dense through the use of polyester fibers or polyester fabric to adequately seal gaps 200 between heart valve 100 and native valve annulus 250 without the need for major tissue growth at gaps 200. When foam 300 is functioning properly, heart valve 100 will be adequately sealed within native valve annulus 250 so that blood flows through valve assembly 104, while limiting or at least reducing blood flow through any gaps formed between the heart valve and the native valve annulus.

While the inventions herein have been described for use in connection with heart valve stents having a particular shape, the stent could have different shapes, such as a flared or conical annulus section, a less-bulbous aortic section, and the like, and a differently shaped transition section. Additionally, though biocompatible foam 300 has been described for use in connection with expandable transcatheter aortic valve replacement, it may also be used in connection with surgical valves, sutureless valves and other devices in which it is desirable to create a seal between the periphery of the device and the adjacent body tissue. Although the deployment of biocompatible foam 300 has been described using a catheter that deploys prosthetic heart valve 100 in tandem with ring 330, it will be understood that the heart valve may be delivered first, followed by the foam ring. It will also be understood that while the preceding disclosure has illustrated the use of a single foam ring 330 to fill gaps, multiple foam rings and other structures may be deployed at varying lateral sections of a heart valve.

Moreover, although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

In some examples, the biocompatible foam may include a polymeric shape-memory material. The biocompatible foam may include a heat-activated shape-memory material. The shape-memory material may have a transition temperature of between about 34 degrees Celsius and about 40 degrees Celsius. The shape-memory material may have a transition temperature that is within one degree Celsius from core body temperature of a human being. The body may include a plurality of voids dispersed throughout its mass to reduce density. The body may have a density of between about 10 kg/m³ and about 60 kg/m³. The body, when fully expanded may have a circumferential stress of about 350 kPa. The body may be compressible to fit within a 18 Fr delivery system. The medical device may be a prosthetic heart valve having a collapsible and expandable stent, a valve assembly disposed in the stent for controlling the flow of blood through the stent, and a cuff disposed about the valve assembly, and wherein the body overlaps with a portion of the cuff. The body may be configured to radially project about 3 mm to about 5 mm from an outer circumference of the medical device when no external force is applied thereupon. The body may be coupleable to the medical device via sutures.

In some examples, the biocompatible foam may include a polymeric shape-memory material. The biocompatible foam may include a heat-activated shape-memory. The body may be compressed within the sheath and self-expands upon unsheathing. The body may include a biocompatible foam. The biocompatible foam may include a heat-activated shape-memory polymer that changes shape when deployed at the target site. Additionally, in certain methods, sealing may be encouraged by flushing the body with a fluid to aid in expanding the biocompatible foam of the body.

It will be appreciated that the various dependent claims and the features set forth therein can be combined in different ways than presented in the initial claims. It will also be appreciated that the features described in connection with individual embodiments may be shared with others of the described embodiments.

1. A biocompatible foam structure for sealing a gap between a medical device and adjacent body tissue, the structure comprising:
an expandable ring-shaped body configured to be disposed about the medical device and to expand from a first radius in a compressed condition to a second radius in an expanded condition, the second radius being greater than the first radius, the body being formed from a compliant biocompatible configured and arranged to conform to the body tissue.

2. The biocompatible foam structure of claim 1, wherein the biocompatible foam comprises a polymeric shape-memory material.

3. The biocompatible foam structure of claim 2, wherein the biocompatible foam comprises a heat-activated shape-memory material.

4. The biocompatible foam structure of claim 3, wherein the shape-memory material has a transition temperature of between about 34 degrees Celsius and about 40 degrees Celsius.

5. The biocompatible foam structure of claim 3, wherein the shape-memory material has a transition temperature that is within one degree Celsius from core body temperature of a human being.

6. The biocompatible foam structure of claim 1, wherein the body includes a plurality of voids dispersed throughout its mass to reduce density.

7. The biocompatible foam structure of claim 1, wherein the body has a density of between about 10 kg/m³ and about 60 kg/m³.

8. The biocompatible foam structure of claim 1, wherein the body, when fully expanded has a circumferential stress of about 350 kPa.

9. The biocompatible foam structure of claim 1, wherein the body is compressible to fit within a 18 Fr delivery system.

10. The biocompatible foam structure of claim 1, wherein the medical device is a prosthetic heart valve having a collapsible and expandable stent, a valve assembly disposed in the stent for controlling the flow of blood through the stent, and a cuff disposed about the valve assembly, and wherein the body overlaps with a portion of the cuff.

11. The biocompatible foam structure of claim 1, wherein the body is configured to radially project about 3 mm to about 5 mm from an outer circumference of the medical device when no external force is applied thereupon.

12. The biocompatible foam structure of claim 1, wherein the body is coupleable to the medical device via sutures.

13. A prosthetic heart valve, comprising:
a collapsible and expandable stent having a proximal end, a distal end, an annulus section adjacent the proximal end and an aortic section adjacent the distal end;
a collapsible and expandable valve assembly disposed within the stent and including a plurality of leaflets;
a cuff annularly disposed about the valve assembly in the annulus section; and
a ring-shaped body disposed about the stent and configured to expand from a first radius in a compressed condition to a second radius in an expanded condition, the second radius being greater than the first radius, the body being formed from a compliant biocompatible foam configured to conform to body tissue.

14. The prosthetic heart valve of claim 13, wherein the biocompatible foam comprises a polymeric shape-memory material.

15. The prosthetic heart valve of claim 14, wherein the biocompatible foam comprises a heat-activated shape-memory material.

16. A method of sealing a space between a medical device and adjacent tissue, the method comprising:
delivering the medical device and a ring-shaped body to a target site using a delivery system having a sheath disposed over the medical device and the body;
removing the sheath to expose the medical device and the body such that the medical device and the body expand; and
sealing the space between the expanded medical device and the adjacent tissue with the expanded body disposed therebetween.

17. The method of claim 16, wherein the body is compressed within the sheath and self-expands upon unsheathing.

18. The method of claim 17, wherein the body comprises a biocompatible foam.

19. The method of claim 18, wherein the biocompatible foam comprises a heat-activated shape-memory polymer that changes shape when deployed at the target site.

20. The method of claim 19, wherein the sealing step includes flushing the body with a fluid to aid in expanding the biocompatible foam of the body.
